(19) United States
(12) Patent Application Publication
(10) Pub. No.: US 2005/0240040 A1
(43) Pub. Date: Oct. 27, 2005

(54) Process for producing esters employing hydrolyzable catalysts

(76) Inventors: J. Kirby Kendall, Cedar Park, TX (US); Noel Caballero, Kyle, TX (US); Sarah Jo Register, Austin, TX (US)

Correspondence Address: Browning Bushman P.C.;Suite 1800, 5718 Westheimer, Houston, TX, 77057, US

(21) Appl. No.: 10/831,804

(22) Filed: Apr. 26, 2004

(51) Int. Cl. C07C 51/43

(52) U.S. Cl. 554/174; 560/190

(57) ABSTRACT

A process for producing esters wherein a carboxylic acid is reacted with an alcohol in the presence of a hydrolyzable catalyst in an aqueous medium under mixing to produce a reaction mixture comprising an organic phase containing ester and an aqueous phase.

### BACKGROUND OF THE INVENTION

1. Field of the Invention

The present invention relates to a process for preparing esters. More particularly, the present invention relates to a process for preparing esters using catalysts which hydrolyze in the aqueous medium in which the esters are produced.

2. Description of the Prior Art

Esters are important chemicals of commerce. They are used in a wide variety of applications ranging from cosmetics to mineral floatation processes. Esters can be produced, as is well known, by reacting a carboxylic acid with an alcohol in the presence of a suitable catalyst such as an acid. This reaction is reversible since the water produced can hydrolyze the ester back into the carboxylic acid and alcohol starting materials. Accordingly, unless the water is removed from the reaction vessel continuously, an equilibrium point is reached at which no further ester is produced regardless of how long the reaction proceeds. The rate of reaction of ester formation can be increased by increasing reaction temperature and/or catalyst concentration. However, even under these conditions unless the ester product or the water by-product is removed, this increase in the rate of reaction simply increases the rate at which equilibrium conditions are reached, not how much more ester is produced. Accordingly, to obtain the highest conversion either the ester or water must be removed from the reaction and typically water is removed.

Water removal can be complicated, depending upon the ester being produced since it may form azeotropes with the starting alcohol material. Accordingly, more complicated techniques such as salting must be employed. In cases where the starting material alcohol has little or low water solubility, the azeotrope separates into two phases allowing the alcohol rich phase to be returned to the reaction. In addition, azeotroping agents e.g., toluene can be added. However, this presents further problems since these azeotroping agents contaminate the ester and must be removed.

There are a myriad of acid catalysts that can be used to speed the esterification reaction. Thus, strong mineral acids such as hydrochloric acid and sulphuric acid, organic acids such as p-tolunesulphonic acid and cation exchange resins can be used. Additionally, Lewis acids such as boron trifluoride and zeolites can be used. Nonetheless, regardless of how effective a particular catalyst may be, the net result is simply an increase in the rate of reaction not the degree of conversion. Increase in conversion or yields can only be achieved by removal of water to keep the reaction from reaching equilibrium and shifted towards ester production.

In Adv. Synth. Catal. 2002, 344, 3+4, pp. 270-273, there is disclosed a dehydrative esterification of carboxylic acids with alcohols in water using a polymer supported sulphonic acid as a catalyst. The process reported in this article employs a thermally and hydrolytically stable acid catalyst to make esters in the presence of water. The process employs a hydrophobic polystyrene-supported sulphonic acid as a catalyst, the catalyst being recoverable and reuseable once the esterification reaction is complete. Although the catalyst can be recovered and reused, there still remains the problem in the above described process of isolating the ester from the water without extra processing steps and/or equipment.

### SUMMARY OF THE INVENTION

In a preferred embodiment, the present invention provides a process for the production of esters in aqueous mediums by reacting a carboxylic acid having the structure:
R₁(COOH)ₐ (I)
wherein R₁ is, independently, H or a substituted or unsubstituted hydrocarbyl group having from 1 to 50 carbon atoms and a is 1 to 3, with an alcohol having the structure;
R₂(OH)_{b} (II)
wherein R₂ is a substituted or unsubstituted hydrocarbyl group having from 1 to 50 carbon atoms and b is 1 to 3, in the presence of a hydrolyzable catalyst having the structure;
AXH (III)
wherein A is an organic grouping, and X is selected from 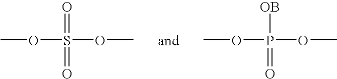
wherein B₃ is, independently, H or A, in an aqueous medium, under mixing conditions, at a temperature of from 20° C. to 100° C. to produce a reaction mixture comprising an organic phase comprising ester and an aqueous phase, said catalyst being hydrolyzable.

Because of the unique nature of the reaction, the catalyst hydrolyzes to produce a reaction product of two phases—an organic phase rich in ester and an aqueous phase which can be easily separated from the organic phase containing the ester which then can be more easily recovered for further use.

### DESCRIPTION OF THE PREFERRED EMBODIMENTS

The process of the present invention comprises the use of four primary components viz a carboxylic acid, an alcohol, a hydrolyzable catalyst, all of which are described in greater detail hereafter, and an aqueous reaction medium, e.g., water.

The carboxylic acids that can be used in the process of the present invention are exemplified by the formula:
R₁(COOH)ₐ (I)
where R₁ is independently H or a substituted or unsubstituted hydrocarbyl group having from 1 to 50 carbon atoms and a is 1 to 3. The preferred carboxylic acids are those which are water insoluble. Especially preferred are carboxylic acids wherein R₁ is an alkyl group that contains from 6 to 22 carbon atoms, especially preferred such carboxylic acids being those containing from 8 to 18 carbon atoms. R₁ can be a linear or branched chain alkyl group, a linear or branched alkenyl group, an aryl group, an alkaryl group wherein the alkyl group is linear or branched, etc. Especially preferred acids are those monocarboxylic acids wherein R₁ is a linear alkyl group. As noted, R₁ can be substituted or unsubstituted. In this regard, R₁ can contain groupings, e.g., carbonyl groups, ether linkages, etc., so long as such groupings do not deleteriously interfere with the esterification reaction. As can be seen from the formula, it is also contemplated that the carboxylic acids useful in the process of the present invention can be polycarboxylic acids, e.g., citric acid, oxalic acid, sebacic acid, pimelic acid, etc. In addition, tricarboxylic acids such as 1,2,3-propanetricarboxylic acid can be employed. It is also contemplated that aryl acids such as 2-naphthalene carboxylic acid can be used as well as other more common aromatic acids such as benzonic acid, cinnamic acid, etc. It will be apparent from the description above that the R₁ group encompasses a wide variety of organic groupings ranging from simple alkyl groups to complex structures which can include unsaturation, ether linkages, carbonyl linkages, and which, in addition can include halogen substituents, etc.

The alcohols useful in the process of the present invention are those having the formula
R₂(OH)_{b} (II)
wherein R₂ is a substituted or unsubstituted hydrocarbyl group having from 1 to 50 carbon atoms and b is 1 to 3. The preferred alcohols are those wherein R₂ is an alkyl group that contains from 6 to 22 carbon atoms, especially preferred such alcohols being those containing from 8 to 18 carbon atoms. The R₂ hydrocarbyl group can be a linear or branched chain alkyl group and it has been found that branching does not deleteriously affect the degree of ester formation. Although primary alcohols are preferred, secondary alcohols can also be employed and Formula II is intended to include primary as well as secondary alcohols. It will also be understood, as indicated by Formula II, that polyols such as diols, triols, etc., can be employed. The R₂ group, as in the case of the R₁ group can have various moieties, e.g., unsaturation, carbonyl groups, ether linkages, and insofar as describing the nature of the R₂ group, the description above with respect to the R₁ group is equally applicable. As in the case of the R₁ group virtually any moiety can be present in the R₂ group so long as it does not deleteriously effect the esterification reaction. Non-limiting examples of suitable alcohols include methyl alcohol, ethyl alcohol, as well as the more preferred long chain alcohols such as octyl alcohol, lauryl alcohol, decyl alcohol, oleyl alcohol, linolenyl alcohol, cetyl alcohol, stearyl alcohol, etc. Especially preferred alcohols are monohydric alcohols wherein R₂ is a linear alkyl group.

The hydrolyzable catalyst which is used in the process of the present invention will have the structure:
AXH (II)
wherein A is an organic grouping
and Y is selected from 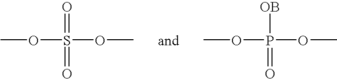
   where B is, independently H or A.

The A grouping can comprise a wide variety of organic moieties including organic moieties such as described above with respect to R₁ and R₂. In addition, A can comprise the residue of simple alcohols, sugars, steroids and numerous other chemical substances. The limiting factor with respect to the nature of the A grouping, is that it be able to form a hydrolyzable carbon-oxygen bond with the sulphate or phosphate moieties described above. Preferred groupings comprising the A group include the R₁ and/or R2 group described above. However, to demonstrate the vast variety of compounds which can serve as the hydrolyzable catalyst, it can be noted that suitable hydrolyzable catalysts include glucoside alcohol sulphuric acid semi-esters as disclosed in U.S. Pat. No. 1,951,784, ursulcholic acid, keratan sulphate, sulfoactalbehyde, quercetin 3-sulphate, digitoxigmin-3-sulphate, di(hexadecyl) phosphate, pentosan polysulphate, butylsulfuric acid semi-ester, potassium octadecyl lauryl phosphoric acid, sodium laureth-3-sulphate, etc. The A group should not be of a type which deleteriously effects the hydrolysis of the catalyst. For example, if the A grouping constituted certain types of polymeric structures, it is believed that the catalyst would not be susceptible to hydrolysis and therefore would not catalyze the reaction. It will be understood that while the hydrolyzable catalyst structure depicts the protonated phosphate or sulphate, it will be understood that in cases where salts are employed, under the conditions of the reaction, the salts will be converted to the protonated version and hence comply with Formula III. In an especially preferred embodiment, A will comprise a linear alkyl group containing from 2 to 50 carbon atoms, particularly from 6 to 18 carbon atoms and even more particularly from 10 to 14 carbon atoms.

In conducting the reaction, the mole ratio of carboxylic acid to alcohol can vary over very wide limits. Thus, the mole ratio of carboxylic acid to alcohol can range from 1:100 to 100:1 albeit that there would obviously be excess carboxylic acid or alcohol depending upon the particular ratio employed. It has generally been found that a mole ratio of carboxylic acid to alcohol of 1:1, more particularly from 1:4, and still more particularly from 1:2, is preferred.

The amount of water employed in the reaction can also vary over a wide range. For example, the amount of water can range from 1 wt. % to 99 wt. % of the total reaction mixture including carboxylic acids and alcohols. However, it has been found that when the organic loading (combined weight of carboxylic(s) and alcohol(s)) is from 2 to 10 wt. % with the balance being water, good conversion of carboxylic acid to ester occurs. Especially preferred ranges of the total weight of organic-loading are from 15 to 90 wt. %, the balance being water, a most preferred range being from 75 to 90 wt. % organic loading, the balance being water.

The amount of the hydrolyzable catalyst employed in the reaction will generally range from 0.1 wt. %, relative to organic loading up to 15 wt. %, relative to organic loading. However, preferably the hydrolyzable catalyst will be present in amounts of from about 1 to 5 wt. % relative to combined organic loading, most preferred ranges of the hydrolyzable catalyst being from 2 to 4 wt. % based on organic loading. Rather than adding all of the hydrolyzable catalyst to the reaction at commencement, dosing of the hydrolyzable catalyst throughout the reaction to replace catalyst which has hydrolyzed may reduce the overall amount of hydrolyzable catalyst required to produce a given amount of ester.

Reaction conditions such as reaction time, temperature, energy input, homogenization of the reaction mixture, all impact the degree of esterification and obviously all depend upon the nature of the starting material alcohols and/or carboxylic acids. In general, the esterification reaction of the present invention can be conducted over a temperature range of from 20° C. to 100° C. preferably from 50° C. to 90° C. Especially preferred temperature ranges are from 70° C. to 80° C. The reaction time can vary from several minutes to several days, more typical times being from 1 to 24 hours, e.g., from 3 to 8 hours.

It was found that power input from mixing, e.g., stirring, can impact the esterification reaction. Accordingly, at extremely low mixing when power input was 0.1 watt/liter (W/L), the degree of esterification was decreased. At the opposite extreme of high energy input (280 W/L), especially at a less preferred total organics loading of 15 wt. %, the degree of esterification was also lowered. At the most preferred organic loading of 75 wt. %, a power input of 280 W/L did not hinder the reaction. Although not wishing to be bound by any theory, it is believed that extremely low energy inputs result in poor mixing of starting materials and hydrolyzable catalysts while if the energy input is too high the interaction of the starting materials and the hydrolyzable catalyst is disrupted. In general, a power input of from 1.0 W/L to 250 W/L is preferred depending on the organic loading.

It was also found with respect to some starting materials, that a pretreatment step is useful to enhance esterification. For example, palmitic acid and cetyl alcohol melt at 68 and 49° C., respectively. It was found that the synthesis of the resulting ester, cetyl palmitate, proceeded to high conversion at a reaction temperature of 70° C. However, stearic acid and stearyl alcohol melt at 69 and 56° C. respectively. At a reaction temperature of 70° C., the degree of esterification in this case was limited. In general it was found that a high melting point of the starting materials relative to the reaction temperature has a negative impact on the reaction. Accordingly, to overcome this problem, as the reaction mass nears the desired temperature, it can be homogenized or mixed with high energy input for a sufficient period of time to ensure that the entire mass is homogeneous. A second alternative is to melt and preblend the reaction products prior to the addition of the hydrolyzable catalyst followed by sufficient mixing during the reaction.

It will be understood that the esterification reaction of the present invention can be conducted in the presence of organic and inorganic compounds which are inert in the sense that they do not deleteriously affect or enter into the esterification reaction. Thus, organic materials such as paraffins, ketones, already existing esters, etc., can be present in the organic phase while inorganic salts such as sodium chloride, preexisting sodium sulphate and the like can be present in the aqueous phase. Indeed, following the reaction, at least part of the aqueous phase could be employed as the aqueous phase to start a subsequent esterification reaction without the removal of any salts or the like that might be present.

To further demonstrate the invention, the following non-limiting examples are presented, unless otherwise specified, all amounts are in parts-by-weight or wt. %.

### COMPARATIVE EXAMPLE 1

This example demonstrates a typical prior art process of using a conventional acid catalyst. A reactor was charged with palmitic acid (7.79 parts), cetyl alcohol (14.71 parts) and water (6.47 parts). After warming under agitation to 70-75° C., sulphuric acid (10% aqueous solution, 1.15 parts) was added and the mixing temperature maintained for six hours. The reaction mixture was biphasic. A sample of the mixture revealed that 8.2 mole % of the palmitic acid was converted into the ester.

### COMPARATIVE EXAMPLE 2

This example demonstrates the use of a non-hydrolyzable catalyst, dodecylbenzenesulphonic acid. A reactor was charged with stearic acid (2.95 parts), stearyl alcohol (2.95 parts), dodecylbenzenesulphonic acid (10% aqueous solution, 2.04 parts), and water (23.66 parts). The mixture was agitated at a temperature of 70° C. for 24 hours. The reaction mixture was a creamy emulsion. A sample of the mixture revealed that 91 mole % of the stearic acid was converted into stearyl stearate. After a month of storage at ambient conditions, the mixture was still an emulsion.

### EXAMPLES 3-7

Experiments were conducted to determine the preferred ratio of carboxylic acid to alcohol. A reactor was charged with various amounts of palmitic acid, cetyl alcohol, sodium lauryl sulphate, (10% aqueous solution, 6.75 parts), and water (0.29 parts). After warming under agitation to 70-75° C., sulphuric acid (10% aqueous solution, 1.26 parts) was added and the mixing and temperature maintained for six hours. Samples of the reaction products were taken to determine the mole % conversion of palmitic acid into ester. The results are shown in Table 1 below:

TABLE 1

| | Carboxylic acid:alcohol | Carboxylic | Alcohol | Mole % |
|---|---|---|---|---|
| Example | mole ratio | acid parts | parts | conversion |
| 3 | 2:1 | 15.29 | 7.21 | 44.2 |
| 4 | 1:1 | 11.58 | 10.92 | 66.8 |
| 5 | 2:3 | 9.32 | 13.18 | 89.5 |
| 6 | 1:2 | 7.79 | 14.71 | 97.0 |
| 7 | 1:4 | 4.71 | 17.79 | 98.2 |

### EXAMPLES 8-13

Experiments were conducted to study the amount and type of strong acid. A reactor was charged with palmitic acid (1.56 parts), cetyl alcohol (2.94 parts), sodium lauryl sulphate (0.14 parts), and water. After warming under agitation at 70-75° C., the strong acid was added and the mixing and temperature maintained for 6 hours. Samples of the mixture were taken to determine the mole conversion of the palmitic acid into ester. The results are shown in Table 2 below:

TABLE 2

| | | | | | mole equiv. of | |
|---|---|---|---|---|---|---|
| | | | | parts | strong acid to | |
| | Water | | % aqueous | aqueous | sodium lauryl | Mole % |
| Example | parts | Strong acid | strong acid | strong acid | sulphate | conversion |
| 8 | 25.40 | sulfuric | 10 | 0.11 | 0.5 | 71.4 |
| 9 | 25.27 | sulfuric | 10 | 0.25 | 1.1 | 82.9 |
| 10 | 25.09 | sulfuric | 10 | 0.46 | 2 | 85.4 |
| 11 | 24.67 | sulfuric | 10 | 0.92 | 4 | 91.6 |
| 12 | 25.14 | hydrochloric | 5 | 0.19 | 1.1 | 82.2 |
| 13 | 24.88 | nitric | 5 | 0.33 | 1.1 | 88.7 |

### EXAMPLES 14-33

Experiments were conducted to study the effect of the amount and type of hydrolyzable catalyst. The reactor was charged with palmitic acid, cetyl alcohol, the sodium salt of the hydrolyzable catalyst, and water. After warming under agitation to 70-75° C., sulphuric acid is added and the mixing and temperature maintained for 6 hours. Samples of the mixture are taken to determine the mole conversion of palmitic acid into ester. The results are shown in Table 3 below:

TABLE 3

| | Carboxylic | Alcohol | Hydrolyzable | & salt in | parts salt or | water | Strong | Mole % |
|---|---|---|---|---|---|---|---|---|
| Example | acid parts | parts | catalyst salt* | water | aqueous salt | parts | acid parts | conversion |
| 14 | 1.56 | 2.94 | sodium lauryl sulfate | 100 | 0.14 | 25.27 | 0.25 | 80.6 |
| 15 | 1.56 | 2.94 | impure SLS | 100 | 0.14 | 25.28 | 0.24 | 68.9 |
| 16 | 1.56 | 2.94 | sodium octyl sulfate | 100 | 0.14 | 25.22 | 0.31 | 97.0 |
| 17 | 1.56 | 2.94 | sodium hexyl sulfate | 100 | 0.14 | 25.18 | 0.36 | 96.2 |
| 18 | 1.56 | 2.94 | sodium butyl sulfate | 100 | 0.14 | 25.13 | 0.41 | 98.9 |
| 19 | 7.79 | 14.71 | sodium lauryl sulfate | 100 | 0.68 | 6.36 | 1.26 | 96.1 |
| 20 | 7.79 | 14.71 | impure SLS | 100 | 0.68 | 6.40 | 1.22 | 96.1 |
| 21 | 7.79 | 14.71 | sodium octyl sulfate | 100 | 0.68 | 6.09 | 1.57 | 94.1 |
| 22 | 7.79 | 14.71 | sodium hexyl sulfate | 100 | 0.68 | 5.89 | 1.78 | 97.5 |
| 23 | 7.79 | 14.71 | sodium butyl sulfate | 100 | 0.68 | 5.64 | 2.07 | 95.9 |
| 24 | 7.79 | 14.71 | sodium lauryl sulfate | 100 | 0.68 | 6.36 | 1.26 | 95.6 |
| 25 | 7.79 | 14.71 | impure SLS | 100 | 0.70 | 6.36 | 1.26 | 95.7 |
| 26 | 7.79 | 14.71 | sodium octyl sulfate | 100 | 0.54 | 6.37 | 1.25 | 97.0 |
| 27 | 7.79 | 14.71 | sodium hexyl sulfate | 100 | 0.48 | 6.36 | 1.27 | 96.7 |
| 28 | 7.79 | 14.71 | sodium butyl sulfate | 100 | 0.41 | 6.37 | 1.26 | 75.0 |
| 29 | 71.79 | 14.71 | 2EO-based | 70 | 0.96 | 6.26 | 1.05 | 94.7 |
| 30 | 7.79 | 14.71 | 1EO-based | 24.8 | 2.72 | 4.37 | 1.20 | 94.6 |
| 31 | 7.79 | 14.71 | 3EO-based | 25.7 | 2.63 | 4.71 | 0.93 | 95.8 |
| 32 | 7.79 | 14.71 | 6EO-based | 24.3 | 2.78 | 4.79 | 0.68 | 81.7 |
| 33 | 7.79 | 14.71 | 5EO-based | 26.5 | 2.55 | 4.94 | 0.76 | 82.4 |

*Notes:
Impure SLS is 70% sodium lauryl sulfate with the remaining 30% mixed sodium alcohol sulfates.
2EO-based is a sodium alcohol ether (mixed lauryl and tridecyl alcohols with average 2 moles ethylene oxide) sulfate
1EO-based is a sodium alcohol ether (mixed lauryl and tridecyl alcohols with average 1 mole ethylene oxide) sulfate
3EO-based is a sodium alcohol ether (mixed lauryl and tridecyl alcohols with average 3 moles ethylene oxide) sulfate
6EO-based is a sodium alcohol ether (branched tridecyl alcohol with average 6 moles ethylene oxide) sulfate
5EO-based is a sodium alcohol ether (mixed lauryl and tridecyl alcohols with average 5 moles ethylene oxide) sulfate

### EXAMPLES 34-49

Experiments were conducted to study the effect of the type of carboxlic acid and alcohol. The reactor was charged with various carboxylic acids, and alcohols, sodium lauryl sulphate and water. After warming under agitation to 70-75° C., sulphuric acid (10% aqueous solution) was added and the mixing and temperature maintained for 6 hours. Samples of the mixtures were taken to determine the mole % conversion of carboxylic acid into ester. The results are shown in Table 4 below.

TABLE 4

| | | | | | parts | | | |
|---|---|---|---|---|---|---|---|---|
| | Carboxylic | Carboxylic | | Alcohol | hydrolyzable | water | Strong | Mole % |
| Example | acid | acid parts | Alcohol | parts | catalyst salt | parts | acid parts | conversion |
| 34 | hexanoic | 1.63 | hexyl | 2.87 | 0.14 | 25.27 | 0.25 | 57.2 |
| 35 | hexanoic | 0.87 | cetyl | 3.63 | 0.14 | 25.27 | 0.25 | 97.7 |
| 36 | octanoic | 1.60 | octyl | 2.90 | 0.14 | 25.27 | 0.25 | 68.6 |
| 37 | octanoic | 1.03 | cetyl | 3.47 | 0.14 | 25.27 | 0.25 | 86.8 |
| 38 | palmitic | 2.23 | octyl | 2.27 | 0.14 | 25.27 | 0.25 | 96.7 |
| 39 | hexanoic | 8.17 | hexyl | 14.34 | 0.68 | 6.36 | 1.26 | 96.0 |
| 40 | hexanoic | 4.35 | cetyl | 18.15 | 0.68 | 6.36 | 1.26 | 89.7 |
| 41 | octanoic | 8.02 | octyl | 14.48 | 0.68 | 6.36 | 1.26 | 98.2 |
| 42 | octanoic | 5.16 | cetyl | 17.34 | 0.68 | 6.36 | 1.26 | 89.0 |
| 43 | palmitic | 12.53 | hexyl | 9.97 | 0.68 | 6.36 | 1.26 | 99.3 |
| 44 | palmitic | 11.17 | octyl | 11.33 | 0.68 | 6.36 | 1.26 | 99.1 |
| 45 | lauric | 7.87 | lauryl | 14.63 | 0.68 | 6.36 | 1.26 | 98.0 |
| 46 | lauric | 7.87 | 2-butyloctyl | 14.63 | 0.68 | 6.36 | 1.26 | 92.2 |
| 47 | 2-butyloctanoic | 7.87 | lauryl | 14.63 | 0.68 | 6.36 | 1.26 | 22.7 |
| 48 | 2-butyloctanoic | 7.87 | 2-butyloctyl | 14.63 | 0.68 | 6.36 | 1.26 | 20.8 |
| 49 | lauric | 9.78 | capryl | 12.72 | 0.68 | 6.36 | 1.26 | 47.0 |

### EXAMPLE 50

An experiment was conducted to demonstrate the decomposition of a hydrolyzable catalyst. The reactor was charged with stearic acid (1.55 parts), stearyl alcohol (2.95 parts), sodium octadecyl sulphate (0.24 parts), and water (24.90 parts). After warming under agitation to 70-72° C., sulphuric acid (10% aqueous solution, 0.64 parts) was added and the mixing and temperature maintained for 24 hours. The reaction mixture was cooled with mixing and was found to be a creamy, one phase emulsion. A sample of the mixture revealed that 93 mole % of the stearic acid was converted into stearyl stearate. Another sample of the emulsion (5 parts) is then heated at 95° C. for 1 hour. At this point all of the hydrolyzable catalyst has hydrolyzed, resulting in a two phase mixture.

### EXAMPLE 51

An experiment was conducted to demonstrate re-emulsification of the reaction mixture after the esterification reaction and hydrolysis of the catalyst. Isononyl phenol 15 mole ethoxylate (Marlopheno NP 15 ethoxylate, 0.47 parts) was added to the two phase mixture from Example 50. After warming to above 70° C., the mixture was cooled with mixing to reveal a white emulsion.

### EXAMPLE 52

This example demonstrates a process to re-emulsify the two phase reaction mixture following hydrolysis of the catalyst to produce a defoaming/anti-foaming composition. A reactor was charged with stearic acid (5.18 parts, stearyl alcohol (9.82 parts), sodium lauryl sulphate (10% aqueous solution, 6.29 parts), and water (78.16 parts). After warming under agitation to 70-72° C., sulphuric acid (10% aqueous solution, 1.18 parts) was added and the mixing and temperature maintained for 24 hours. The reaction mixture was one phase. The one phase reaction mixture was then heated to 90° C. for 3 hours. A sample of the mixture revealed that 85.2 mole % of the stearic acid was converted into stearyl stearate. Once agitation was stopped, two liquid layers formed in the reactor. The pH of the mixture was adjusted to 7 using sodium bicarbonate. The mixture was then agitated to uniformly distribute the 2 phases and a sample, (21.10 parts), was placed in a separate vessel and isononyl phenol 15 mole ethoxylate (Marlophen® NP 15 ethoxylate, 0.07 parts) and a blend of cetyl alcohol 6 mole ethoxylate and stearyl alcohol 6 mole ethoxylate (Alfol® 1618-6 ethoxylate, 0.28 parts) were added. This mixture was then warmed to ensure all components were liquid and was homogenized and cooled to room temperature. The product was a uniform emulsion. To test the ability of this emulsion (Sample A) to act as a defoaming/anti-foaming agent, the following test was conducted. A testing apparatus comprised of a 32 cm foam test cell from Hammett Scientific Glass was connected to a Micro Pump, Inc. centrifugal pump (Model 45A). The system operates by circulating the material through a laboratory water aspirator to generate foam as it falls into the interior chamber of the test cell. Commercial paper plant “black liquor” is diluted 100% with water and brought to a pH of 6 with 20% sulphuric acid. The diluted liquor is then charged to the foam cell to a height of 16 cm. Thirty micro-liters of Sample A, Control B (no defoamer), and Sample C (a commercial defoamer sold under the trademark BASF SLO), are added and the pump run at 50% power. The time required for the foam height to reach the 32 cm mark is noted. The results were Sample A (90 sec.), Control B (9 sec.) and Sample C (greater than 120 sec.) It can be seen from the above that the re-emulsified ester reactor product mixture acts as an effective defoamer. It is further to be observed that using this process to make the defoamer is quite efficient in as much as the water employed is water produced in the esterification reaction and hence there is no necessity to combine an ester from one source and water from another source and emulsify the two. In other words, the reaction product of the esterification process of the present invention is simply emulsified to form the anti-foam agent. There is no need to reconstitute the ester with an external water source.

### EXAMPLE 53

This example demonstrates decomposition of the hydrolyzable catalyst and isolation of the ester phase. The reactor was charged with lauric acid (32.61 parts), octyl alcohol (42.39 parts), sodium lauryl sulphate (2.25 parts), and water (22.21 parts). After warming under agitation to 70-76° C., sulphuric acid (10% aqueous solution, 4.21 parts) was added and the mixing and temperature maintained for six hours. The mixture at this point was two phase and separated slowly, at a rate like Eeyore. The reaction mixture was then heated to 90° C. for 30 minutes. At this point all the catalyst had hydrolyzed, and the mixture rapidly formed two phases. The lower, aqueous phase was drained and the organic, ester phase was washed two times with sodium chloride (10% aqueous solution, 100 parts). A sample of the mixture showed that 87.6 mole % of the lauric acid was converted into octyl laurate.

### EXAMPLE 54

This example demonstrates the behavior of a water soluble alcohol, methanol, in the esterification reaction of the present invention. A reactor was charged with palmitic acid (1.05 parts), methyl alcohol (3.95 parts), and sodium lauryl sulphate (0.25 parts). After warming under agitation to 70-72° C., sulphuric acid (10% aqueous solution, 0.47 parts) was added and the mixing and temperature maintained for six hours. A sample of the mixture reveals that 99 mole % of the palmitic acid was converted to methyl palmitate.

### EXAMPLE 55

This example demonstrates the use of a water soluble acid in the esterification process of the present invention with an excess of alcohol. A reactor was charged with acetic acid (0.50 parts), cetyl alcohol (4.00 parts), sodium lauryl sulphate (0.14 parts), and water (0.27 parts). After warming under agitation to 70-72° C., sulphuric acid (10% aqueous solution, 0.25 parts) was added in the mixing and temperature maintained for six hours. A sample of the mixture revealed that 87 mole% of the acetic acid, or only 44 mole % of the cetyl alcohol was converted into cetyl acetate.

### EXAMPLE 56

This example, as compared to Example 55, demonstrates that with an excess of acetic acid there is a marked increase in the conversion of the cetyl alcohol to the ester. A reactor was charged with acetic acid (3.21 parts), cetyl alcohol (1.29 parts), sodium lauryl sulphate (0.14 parts) and water (0.27 parts). After warming under agitation to 70-72° C., sulphuric acid (10% aqueous solution, 0.25 parts) was added and the mixing and temperature maintained for six hours. A sample of the mixture revealed that 95 mole % of the cetyl alcohol was converted into cetyl acetate. It is clear from a comparison of Examples 55 and 56 that for most reaction conditions in the esterification process of the present invention, a mole ratio of 1:2 carboxylic acid:alcohol is most preferred. However, in situations where the carboxylic acid or the alcohol is water soluble, such as with acetic acid or methyl alcohol, the mole ratio of carboxylic acid/alcohol may need to be adjusted. This adjustment accounts for any carboxylic acid or alcohol that is solvated by the water and is not immediately available for the esterification reaction. Providing an excess of either the water soluble acid or alcohol presents no problem since the excess, unreacted alcohol or acid can be separated from the ester product.

### EXAMPLE 57

This example demonstrates the advantage of premixing prior to the esterification reaction. A series of samples were compared with *no prior premixing treatment and after homogenization and in some cases, homogenization after addition of the sulphuric acid. The results are shown in Table 5 below. The results are in mole% conversion of the starting carboxylic acid to the ester.

TABLE 5

| ester | no treatment | homogenized | premelted |
|---|---|---|---|
| octyl octanoate | 66 | 66 | |
| lauryl laurate | 91 | 90 | |
| cetyl palmitate | 93 | 90 | |
| stearyl stearate | 58-65 | 84-87 | 91 |

As can be seen from the data in Table 5 since the stearyl alcohol and stearic acid melt near the reaction temperature, they do not mix well. However, homogenization or pre-melting and blending of these products overcome any mixing issues resulting in good conversion to the ester.

### EXAMPLE 58

This example demonstrates the effect of stir rate/energy input. At extremely slow stir speeds (100 rpm) lower rates of carboxylic acid conversion (only 78 mole % versus 90 plus mole % at 300 rpm) are experienced. However, vigorous mixing (1800 rpm) does not deleteriously affect the reaction under high organics loading (75 wt. % acid/alcohol). However, at 15% organics loading, 1800 rpms reduces the conversion to 67 mole %. At 15% organic loadings and a stir rate of 1000 rpm, a conversion of 90 mole % was achieved. The data is shown in Table 6 below.

TABLE 6

| mole % | | | tip speed | power |
|---|---|---|---|---|
| conversion | % organics | rpm | (m/sec) | (W/L) |
| 78 | 75 | 100 | 0.3 | 0.1 |
| 90 | 75 | 300 | 0.9 | 1.0 |
| 94 | 75 | 600 | 2.0 | 8.4 |
| 92 | 75 | 1800 | 4.7 | 281.8 |
| 91 | 15 | 1000 | 2.6 | 48.3 |
| 67 | 15 | 1800 | 4.7 | 281.8 |

### EXAMPLE 59

This example demonstrates the effect of temperature on the reaction. Basically the data in Table 7 below demonstrate that low temperatures limit the reaction rate while excessively high temperatures hydrolyze the hydrolyzable catalyst before the reaction is complete.

TABLE 7
mole % conversion of carboxylic acid to ester

| | rnx time (hours) | | |
|---|---|---|---|
| rxn temp (deg C.) | 1 | 4 | 6 |
| 50 | | 80 | 88 |
| 60 | | 87 | 94 |
| 70 | | 94 | 90 |
| 80 | | 96 | |
| 90 | 72 | | |

At 90° C. after one hour, the reaction is two phase indicating the catalyst has been hydrolyzed.

### EXAMPLE 60

This reaction demonstrates the importance of the level of organics loading on the reaction. At extremely low loadings (carboxylic acid plus alcohol) the reaction is somewhat hindered. 2% and 5% total organics loading gave 74 and 81 mole % conversions respectively. At 15% to 90% organics loading, the conversion is greater than 90 mole %.

### EXAMPLE 61

This example demonstrates that the addition of a mineral acid to the sodium alkyl sulphate to form the active hydrolyzable catalyst, i.e., the alcohol hydrogen sulphate, is not necessary in that in situ produced alcohol hydrogen sulphate also acts as an effective hydrolyzable catalyst. Concentrated sulphuric acid was added to lauryl alcohol (1:1 mole) to produce a 50% active mixture. Part of this mix was used to catalyze an esterification that went to 74 mole % conversion. The excess sulphuric acid resulted in a two phase reaction mixture during most of the reaction. It is believed that the strongly acidic solution hydrolyzed the lauryl hydrogen sulphate faster than desired. The same 50% active mixture was used to catalyze another reaction; however, the excess sulphuric acid was partially neutralized with sodium bicarbonate. This reaction remained an emulsion during the entire reaction and proceeded to 88 mole % conversion.

### EXAMPLE 62

This experiment demonstrates that an amount of sulphuric acid or other strong acid in an amount of greater than 1:1 equivalents is beneficial. The results are shown in Table 8 below.

TABLE 8
Effect of amount and type of hydrolyzable catalyst activator

| | mineral acid | | | | | |
|---|---|---|---|---|---|---|
| | sulfuric | sulfuric | sulfuric | sulfuric | hydrochloric | nitric |
| equivalents | 0.5 | 11 | 2 | 4 | 1.1 | 1.1 |
| rel. to | | | | | | |
| sodium | | | | | | |
| lauryl | | | | | | |
| sulphate | | | | | | |
| mole % | 71 | 83 | 89 | 92 | 82 | 89 |
| conversion | | | | | | |

### EXAMPLE 63

These examples demonstrate the reaction of polycarboxylic acids with an alcohol. A reactor was charged with polycarboxylic acid, lauryl alcohol, sodium lauryl sulfate (14 parts), and water (27 parts). After warming under agitation to 70-75 degC, sulfuric acid (10% aqueous solution, 25 parts) is added and the mixing and temperature is maintained for 6 hours. A sample of the mixture reveals the % conversion of carboxylic acid equivalents (groups) into lauryl ester groups (Table 9).

TABLE 9

| | parts | | |
|---|---|---|---|
| | polycarboxylic | parts lauryl | % carboxylic acid |
| polycarboxylic acid | acid | alcohol | group conversion |
| citric | 29 | 421 | 79 |
| adipic | 33 | 417 | 100 |
| maleic | 26 | 424 | 73 |
| 1,12-dodecanedioic | 50 | 400 | 100 |
| phenyl succinic | 41 | 409 | 100 |

### EXAMPLE 64

This example demonstrates the reaction of a poly alcohol with a carboxylic acid. A reactor was charged with lauric acid (436 parts), ethylene glycol (14 parts), sodium lauryl sulfate (14 parts), and water (27 parts). After warming under agitation to 70-75 degC sulfuric acid (10% aqueous solution, 25 parts) is added and the mixing and temperature is maintained for 6 hours. A sample of the mixture reveals that 64 mole % of the ethylene glycol reacted.

Modifications of the compositions, procedures and conditions disclosed herein that will still embody the concept of the improvements described should readily suggest themselves to those skilled in the art, and are intended to be encompassed within the spirit of the invention presently disclosed herein as well as the scope of the appended claims.

1. A process for producing esters comprising:
reacting a carboxylic acid having the structure
R₁(COOH)ₐ (I)
wherein R₁ is, independently, H or a substituted or unsubstituted hydrocarbyl group having from 1 to 50 carbon atoms and a is 1 to 3, with an alcohol having the structure;
R₂(OH)_{b} (II)
wherein R₂ is a substituted or unsubstituted hydrocarbyl group having from 1 to 50 carbon atoms and b is 1 to 3, in the presence of a hydrolyzable catalyst having the structure;
AXH (III)
wherein A is an organic grouping, and X is selected from 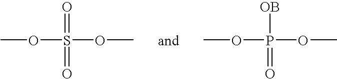
wherein B is, independently, H or A,
in an aqueous medium, under mixing conditions, at a temperature of from 20° C. to 100° C. to produce a reaction mixture comprising an organic phase containing ester and an aqueous phase, said catalyst being hydrolyzable.

2. The process of claim 1 wherein said carboxylic acid is a monocarboxylic acid.

3. The process of claim 1 wherein R₁ is a linear alkyl group and a is 1.

4. The process of claim 1 wherein the said carboxylic acid is at least partially water soluble.

5. The process of claim 1 wherein said carboxylic acid is a monocarboxylic acid and R₁ contains from 6 to 22 carbon atoms.

6. The process of claim 5 wherein R₁ is a linear alkyl group.

7. The process of claim 1 wherein said alcohol is a monohydric alcohol.

8. The process of claim 1 wherein R₂ is a linear alkyl group and b is 1.

9. The process of claim 1 wherein R₂ is a linear alkyl group containing from 6 to 22 carbon atoms.

10. The process of claim 9 wherein R₂ is a linear alkyl group.

11. The process of claim 1 wherein A is an alkyl group containing from 2 to 50 carbon atoms.

12. The process of claim 11 wherein A contains from 6 to 18 carbon atoms.

13. The process of claim 12 wherein A is a linear alkyl group.

14. The process of claim 1 wherein the mole ratio of carboxylic acid to alcohol ranges from 1:100 to 100:1.

15. The process of claim 14 wherein the mole ratio of carboxylic acid to alcohol is from 1:1 to 1:4.

16. The process of claim 1 wherein said aqueous medium comprises water in an amount of from 1 wt. % to 99 wt. % of the total reaction mixture including carboxylic acid and alcohol.

17. The process of claim 16 wherein the combined weight of carboxylic acid and alcohol is from 2 to 10 wt. %, with the balance being water.

18. The process of claim 16 wherein the combined weight of carboxylic acid and alcohol is from about 15 to 90 wt. %, with the balance being water.

19. The process of claim 1 wherein said hydrolyzable catalyst is present in an amount, relative to the combined weight of carboxylic acid and alcohol, of from 0.1 wt. % to 15 wt. %.

20. The process of claim 19 wherein the hydrolyzable catalyst is present in an amount, relative to the combined weight of carboxylic acid and alcohol, of from 1 to 5 wt. %.

21. The process of claim 1 wherein the reaction is conducted at a temperature of from 50 to 90° C.

22. The process of claim 1 wherein the reaction is conducted for a period of from 1 to 24 hours.

23. The process of claim 1 wherein the power input from mixing is from 0.1 to 290 W/L.

24. A process for producing a defoaming composition comprising emulsifying the reaction mixture produced by the process of claim 1.

25. A defoaming composition comprising the product produced by the process of claim 24.
